(19) United States
(12) Patent Application Publication
(10) Pub. No.: US 2025/0082459 A1
(43) Pub. Date: Mar. 13, 2025

(54) ORTHOPEDIC INSERT APPARATUS, SYSTEM, AND METHODS FOR STABILIZATION OF JOINTS

(71) Applicant: BioMedtrix, LLC, Whippany, NJ (US)

(72) Inventors: Christopher B. Preucil, Ketchum, ID (US); Gregory T. van der Meulen, Hailey, ID (US); Christopher G. Sidebotham, Mendham, NJ (US)

(73) Assignee: BioMedtrix, LLC, Whippany, NJ (US)

(21) Appl. No.: 18/827,323

(22) Filed: Sep. 6, 2024

Related U.S. Application Data
(60) Provisional application No. 63/581,199, filed on Sep. 7, 2023.

(51) Int. Cl. A61F 2/08; A61B 17/04

(52) U.S. Cl. CPC A61F 2/0811; A61B 2017/0409

(57) ABSTRACT

Orthopedic implants, insertion apparatus, and methods of insertion and joint stabilization. Orthopedic inserts disclosed herein are configured for insertion into a bone tunnel and extension of a suture therethrough. The orthopedic insert apparatus, systems, and methods can prevent, limit, or decrease contact between and/or compression of a suture on soft tissues proximate the exit site of a bone tunnel. The orthopedic insert can include an insert body that has a head portion, a shaft portion, and a bore extending through the insert body. In some examples, the head portion can include a bearing surface comprising a curved surface or a curved rim at a first end of the insert body. The shaft portion can include projections extending from its exterior surface that are arranged in rows that are spaced apart along a longitudinal axis. Inserter and/or reamer apparatuses can be utilized with the orthopedic insert.

### CROSS-REFERENCE TO RELATED APPLICATION

This application claims the benefit of U.S. Provisional Patent Application No. 63/581,199, filed Sep. 7, 2023, which is incorporated by reference herein in its entirety.

### FIELD

The present disclosure pertains to orthopedic implants, including, for example, orthopedic inserts for insertion into a bone tunnel and extension of a suture therethrough, and insertion apparatus and methods of insertion of orthopedic implants and joint stabilization.

### BACKGROUND

Many joints, for example knees, hips, elbows and ankles, can develop laxity and instability through ligament and tendon injuries or tears. Restoring stability to the joint may be accomplished using suture materials that are fixed above and below the joint under tension, which can assist in sharing loads on the joint during activities. For example, a suture under tension may be fixed to a first bone disposed on a first side of a joint and to a second bone on a second side of the joint for stabilization thereof. In many joint reconstructions, a suture is passed through a bone tunnel drilled in at least one of the first bone or the second bone. Generally, using the foregoing suture technique for stabilizing a joint will result in the suture exiting the bone tunnel and resting on both bone and soft tissues. Where the suture exits the bone tunnel, the suture can cause wear on the bone during normal use (flexing and extending, rotation, etc.) of the joint. Overtime, in addition to damaging the bone, the suture can become lax or unstable, which can result in instability of the treated joint. Additionally, contact between the suture and the soft tissue proximate the exit site can cause or result in pain to the patient (human or other animals, such as dogs).

### BRIEF DESCRIPTION OF THE DRAWINGS

FIGS. 1A and 1B are respectively illustrations of an exemplary intact knee joint and an exemplary damaged knee joint including a torn ligament.

FIG. 2A is an illustration of an exemplary suture configuration in a lateral suture technique treatment of a knee joint.

FIG. 2B is a top perspective view of a button anchor for use in the suture configuration of FIG. 2A.

FIG. 2C is a side view of an interference screw anchor for use in the suture configuration of FIG. 2A.

FIGS. 2D-2G are illustrations of other exemplary suture techniques for treatment of unstable joints.

FIG. 3A is a perspective view of an exemplary orthopedic insert, in accordance with the present disclosure.

FIGS. 3B-3G are respectively a first side view, a second side view, a front view, a rear view, a top view, and a bottom view of the orthopedic insert of FIG. 3A.

FIGS. 4A and 4B are respectively a side elevation view and a bottom plan view of the orthopedic insert of FIG. 3A.

FIG. 4C is a cross-sectional view of the orthopedic insert of FIG. 3A taken along the line C-C shown in FIG. 4B.

FIG. 5 is an illustration of an exemplary insertion configuration or state for the orthopedic insert of FIG. 3A within a bone tunnel.

FIG. 6 is an illustration of a first exemplary configuration for a lateral suture technique in which the orthopedic insert of FIG. 3A is partially inserted into a bone tunnel.

FIG. 7 is an illustration of a second exemplary configuration for a lateral suture technique in which the orthopedic insert of FIG. 3A is fully inserted into a bone tunnel.

FIG. 8 is a perspective view illustrating a suture-bearing surface of the orthopedic insert of FIG. 3A.

FIG. 9 is a side view of another exemplary orthopedic insert, in accordance with the present disclosure.

FIG. 10 is a side view of yet another exemplary orthopedic insert, in accordance with the present disclosure.

FIG. 11 is a side view of still another exemplary orthopedic insert, in accordance with the present disclosure.

FIG. 12 is a side view of an exemplary reamer apparatus, in accordance with the present disclosure.

FIGS. 13A and 13B are respectively side elevation and top plan views of the exemplary reamer apparatus of FIG. 12. FIG. 13B illustrates the reamer apparatus before formation of the cutting edges on the cutter.

FIGS. 13C and 13D are cross-sectional views of a cutter of the reamer apparatus of FIG. 12, taken along lines C-C and D-D in FIG. 13A, respectively.

FIGS. 13E and 13F are respectively a top plan view of a distal portion and a top plan view a proximal portion of the reamer apparatus of FIG. 12.

FIG. 14 is a perspective view of an exemplary inserter apparatus, in accordance with the present disclosure.

FIGS. 15A and 15B are respectively side elevation and top plan views of the exemplary inserter apparatus of FIG. 14.

FIG. 15C is a side elevation view of a distal portion of the inserter apparatus of FIG. 14.

FIG. 15D is an end elevation view of a proximal portion of the inserter apparatus of FIG. 14.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

As used in this disclosure and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” “proximal,” “distal,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Unless otherwise indicated, all numbers expressing angles, dimensions, quantities of components, forces, moments, molecular weights, percentages, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing embodiments from discussed prior art, the embodiment numbers are not approximates unless the word “about” is recited.

Although there are alternatives for various components, parameters, operating conditions, etc., set forth herein, that does not mean that those alternatives are necessarily equivalent and/or perform equally well. Nor does it mean that the alternatives are listed in a preferred order unless stated otherwise.

As used herein, values modified by the term “substantially” mean±10% of the stated value. The term “substantially parallel” means an angle of +10° between an object and a reference. In another example, the term “substantially perpendicular” means an angle of 80° to 100° between an object and a reference.

### Overview

The orthopedic inserts and surgical methods in the following examples are described with reference to veterinary orthopedic procedures in canine patients for purposes of illustration. However, the specific examples provided herein are not intended to be limiting, and the orthopedic inserts, reamer and inserter tools, kits, and associated surgical methods of the present disclosure can be adapted for use in a wide variety of species, including in humans.

As discussed above, for repair of an unstable or damaged joint, a suture may be fixed under tension to a first bone disposed on a first side of the joint and to a second bone on a second side of the joint. In some examples, the suture is passed through a bone tunnel in at least one of the first bone or the second bone. At an exit portion or region of the bone tunnel, the tensioned suture can cause wear and abrasion on the bone and/or can cause pain resulting from contact between the suture and soft tissue. Over time, wear on the bone can cause the suture to lose tension and become lax, thereby resulting in loss of stability in the treated joint. Accordingly, there is need for surgical apparatus and methods and techniques that prevent or limit contact between a suture and bone at or proximate the exit portion or region of a bone tunnel, and/or contact between the suture and soft tissue at or proximate the exit portion or region of a bone tunnel.

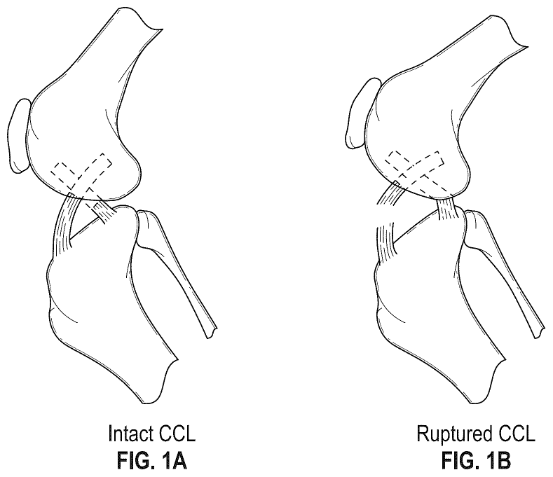

One of the most frequently performed joint stabilization surgical procedures is the lateral suture technique, which can be utilized to repair or stabilize a knee after rupture or tear of a ligament, such as, for example, the cranial cruciate ligament (CCL) in dogs or the anterior cruciate ligament (ACL) in humans. For illustrative purposes, FIGS. 1A and 1B respectively show an exemplary intact (healthy) CCL and an exemplary ruptured (injured) CCL. The lateral suture technique can stabilize a stifle joint (knee joint) having a damaged CCL by affixing (or otherwise anchoring, attaching, fastening, etc.) a first end of a suture to the femur above the joint, passing the suture in a medial to lateral direction through a drilled hole in the femur (i.e., a femoral bone tunnel), pulling the suture taut, and then affixing (or otherwise anchoring, attaching, fastening, etc.) an opposing second end of the suture to the tibia below the joint such that the suture is under tension.

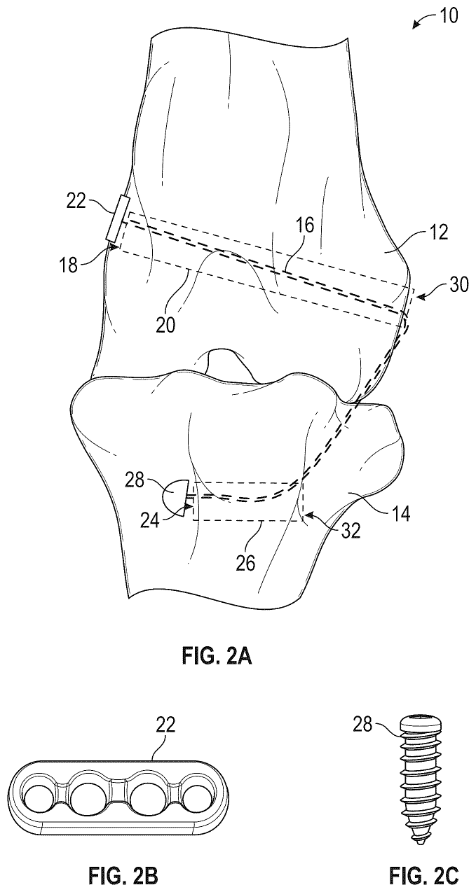

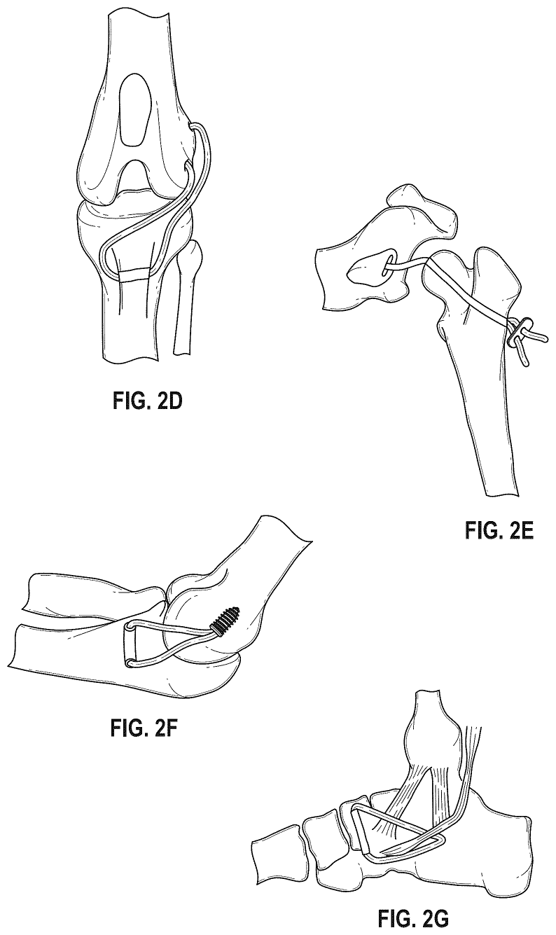

For example, FIG. 2A shows an exemplary suture configuration 10 through a first bone 12 (e.g., a femur) and a second bone 14 (e.g., a tibia) of a canine stifle joint after treatment or reconstruction via a lateral suture technique. In some examples, a first end of a suture 16 can be fixed or secured at an entrance 18 to a femoral bone tunnel 20 utilizing a first anchoring device, such as a button-type anchor 22 (also referred to as a “button”) (FIG. 2B). In some examples, a second (opposing) end of the suture 16 can be fixed or secured at an exit 24 of a tibial bone tunnel 26 utilizing a second anchoring device, such as a screw anchor 28 (e.g., an interference screw) (FIG. 2C).

As can be seen in FIG. 2A, at an exit portion 30 of the femoral bone tunnel 20, a central portion of the suture 16 contacts the bone at the exit and wraps or turns at an acute angle to extend over the surface of a lateral aspect of the femur 12 (e.g., the lateral condyle) toward the tibial bone tunnel 26. Although soft tissues are not illustrated (for clarity), the suture 16 can additionally contact or compress soft tissue proximate the exit portion 30 of the femoral bone tunnel 20. Thus, the suture 16 can cause wear and abrasion on the bone proximate or within the exit portion 30 and/or can cause pain resulting from the contact between the suture and soft tissue proximate or at the exit portion 30.

Alternative suture techniques can be used for stabilization of a knee, for example where the suture can have a different configuration than the configuration 10 of FIG. 2A (such as, for example, the suture configuration shown in FIG. 2D), and/or for stabilization of other joints, such as a hip (for example, the suture configuration shown in FIG. 2E), an elbow (for example, the suture configuration shown in FIG. 2F), or an ankle (for example, the suture configuration shown in FIG. 2G). These alternative suture techniques can have similar issues to those discussed above with respect to the lateral suture technique. For example, each of the foregoing suture techniques can include forming a bone tunnel through a first bone in a joint, affixing a first portion of a suture to the first bone, insertion of the suture through the bone tunnel, pulling the suture taut, and affixing of a second portion of the suture to a second bone of the joint such that the suture is under tension. At locations or regions where the tensioned suture exits the bone tunnel, the suture may over time cause wear or abrasion on the bone and the suture may become lax resulting in instability of the treated joint. Further, the tensioned suture may contact or compress soft tissue at or near an exit location of the bone tunnel, which can cause pain for the patient.

One or more of the foregoing issues associated with suture-based joint stabilization techniques can be addressed via the apparatus, systems, and methods disclosed herein. For example, the orthopedic insert apparatus, systems, and methods disclosed herein can prevent, limit, or decrease abrasion or wear on bone at or proximate a location where a suture exits a bone tunnel in a reconstructed joint. This can facilitate a greater degree of stability in the reconstructed joint over time relative to existing joint reconstruction techniques. In other words, protecting the bone from suture abrasion and wear using the apparatus, systems, and methods disclosed herein can result in a longer-term successful joint reconstruction relative to conventional joint reconstruction techniques.

In another example, the orthopedic insert apparatus, systems, and methods disclosed herein can prevent, limit, or decrease contact between and/or compression of a suture on soft tissues proximate the exit site of a bone tunnel. In this example, patients treated with the orthopedic insert apparatus, systems, and methods disclosed herein may experience reduced pain and/or soft tissue irritation relative to conventional joint reconstruction techniques.

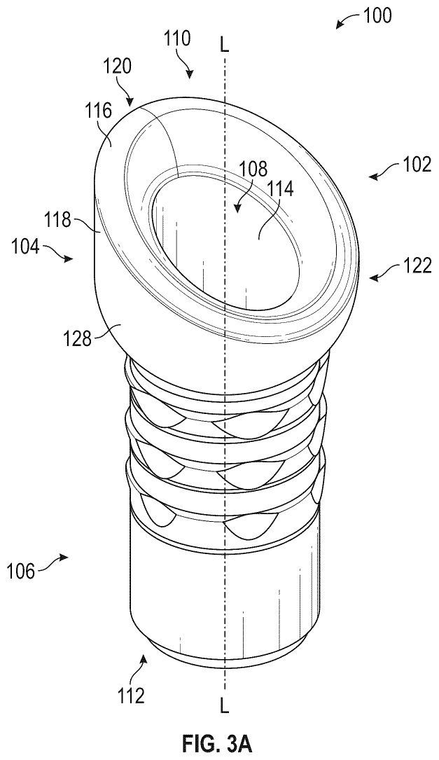

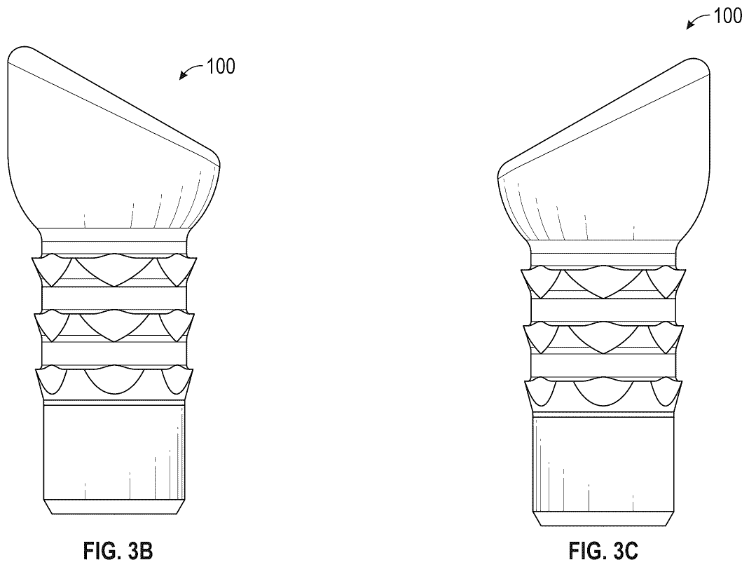

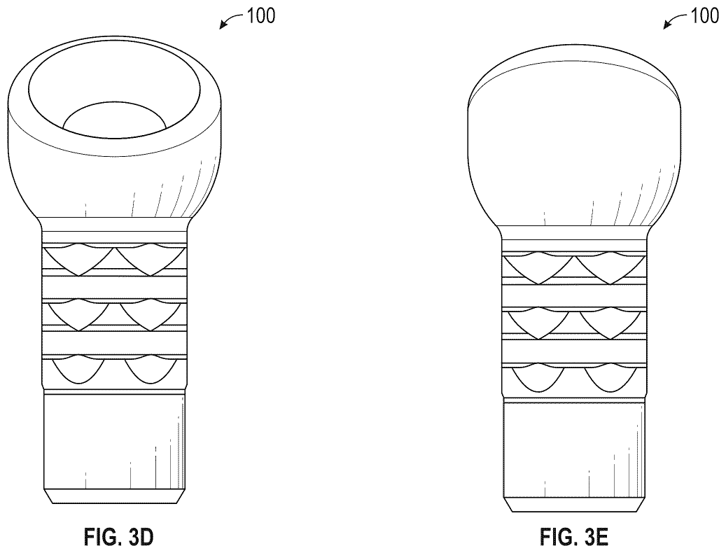

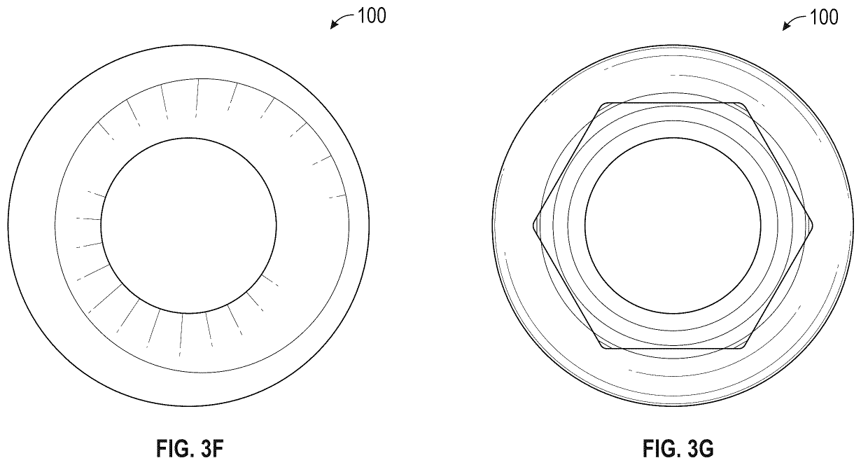

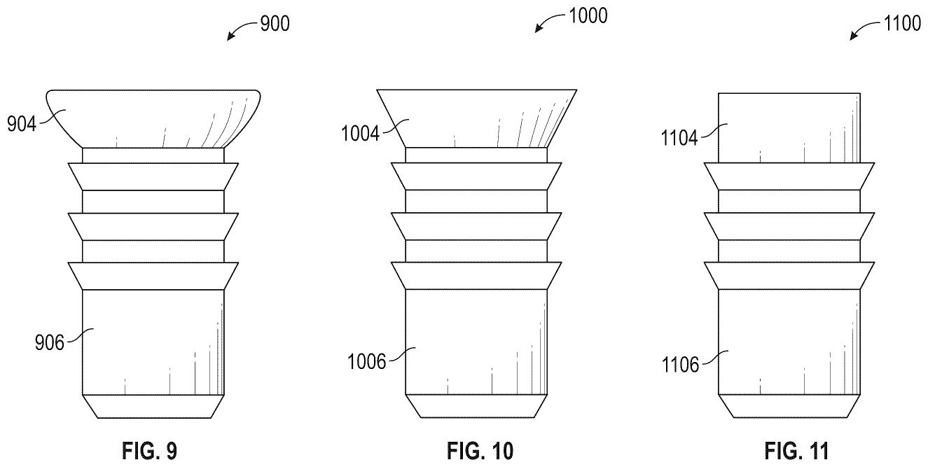

In one representative example, FIGS. 3A-8 illustrate an orthopedic insert including an insert body that has a head portion and a shaft portion extending from the head portion along a longitudinal axis of the insert body. The insert body can include a bore extending therethrough along the longitudinal axis of the insert body. The bore can have a continuous diameter and/or a continuous smooth interior surface. In some examples, the head portion can include a bearing surface comprising a curved surface or a curved rim at a first end of the insert body. In some examples, the rim is a sloped rim that is disposed at an angle relative to the longitudinal axis of the insert body. The rim of the head can thus have an upper or extended portion of the head portion and a lower or base portion of the head portion. In other examples, such as in the exemplary orthopedic inserts of FIGS. 9-11, the head portion can have a different configuration.

In some examples, the insert body can include projections extending from an exterior surface of the shaft. In some examples, the projections can be arranged in rows that are spaced apart along the longitudinal axis of the insert body. In some examples, a height of the projections in a respective row can increase in a direction along the longitudinal axis from a second end of the insert body (e.g., an end of the shaft portion opposite the head portion) toward the first end of the insert body. In some examples, a width of the projections in a respective row can decrease in a direction along the longitudinal axis from the second end of the insert body toward the first end of the insert body.

The orthopedic inserts disclosed herein can be configured for insertion into a bone tunnel in a bone of a joint. For example, an orthopedic insert can be inserted into an exit portion of the bone tunnel. In some examples, the orthopedic insert can be fully inserted into the bone tunnel such that the rim of the head portion is flush or substantially flush with a surface of the bone. In some examples, the orthopedic insert can be partially inserted into the bone tunnel such that the rim of the head portion is offset relative to the surface of the bone (e.g., all or a portion of the head is above or extends outwardly relative to the surface of the bone). After insertion of the orthopedic insert, a first portion of a suture can be affixed or anchored (via, for example, the button shown in FIG. 2B) at an entrance portion of the bone tunnel. The suture can be threaded through the bone tunnel and the bore of the insert body. The suture can be pulled taut and affixed or anchored to a second bone of the joint (via, for example, the interference screw in FIG. 2C) such that the suture is secured under tension.

In each of these examples (examples where the orthopedic insert is fully inserted into the bone tunnel and examples where the orthopedic insert is partially inserted into the bone tunnel), a portion of the suture at the exit portion or region of the bone tunnel of the first bone can contact or extend over the bearing surface of the orthopedic insert and toward a bone tunnel entrance or attachment point in the second bone of the joint. Accordingly, in such examples, the orthopedic insert can limit or prevent contact between the suture and the bone at or proximate to the exit portion of the bone tunnel, and thereby prevent or limit wear or abrasion on the bone during flexing and extending of the treated joint.

Additionally, in examples where the orthopedic insert is partially inserted into the bone tunnel, a portion of the suture at the exit portion or region of the bone tunnel can be offset from the surface of the first bone and extend over the bearing surface toward a bone tunnel entrance or attachment point in the second bone. Accordingly, in such examples, the orthopedic insert can limit or prevent contact between or minimize compression of the suture on soft tissue at or proximate the exit portion of the bone tunnel and thereby prevent, limit, or decrease (relative to conventional suture techniques) pain and soft tissue irritation experienced by the patient after treatment.

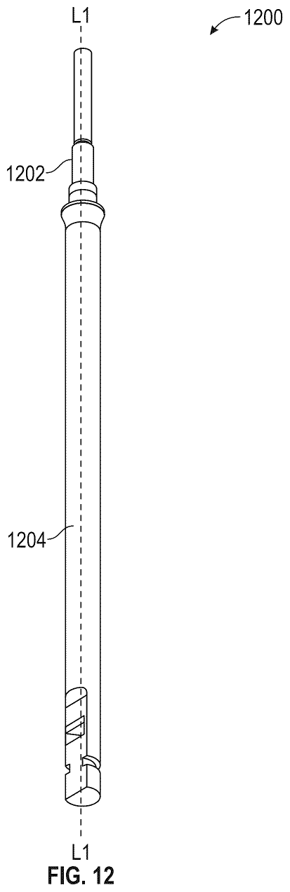

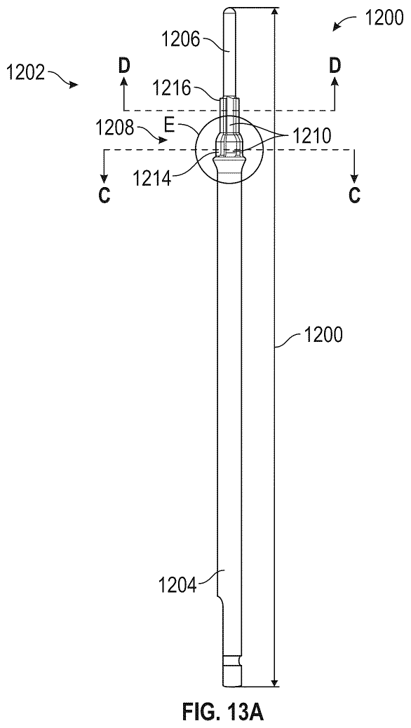

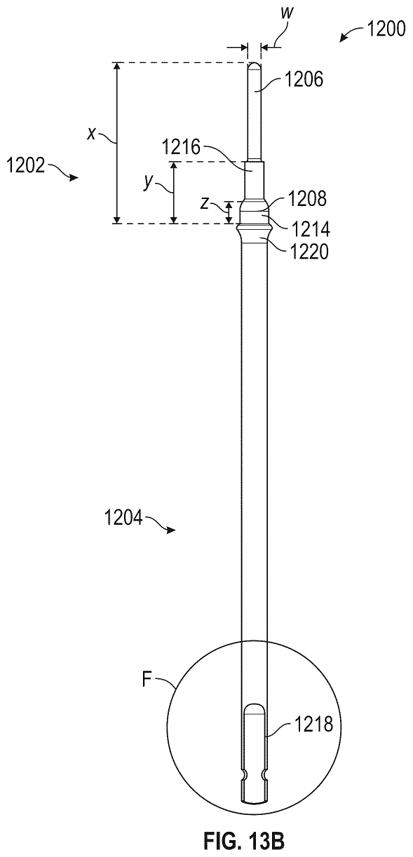

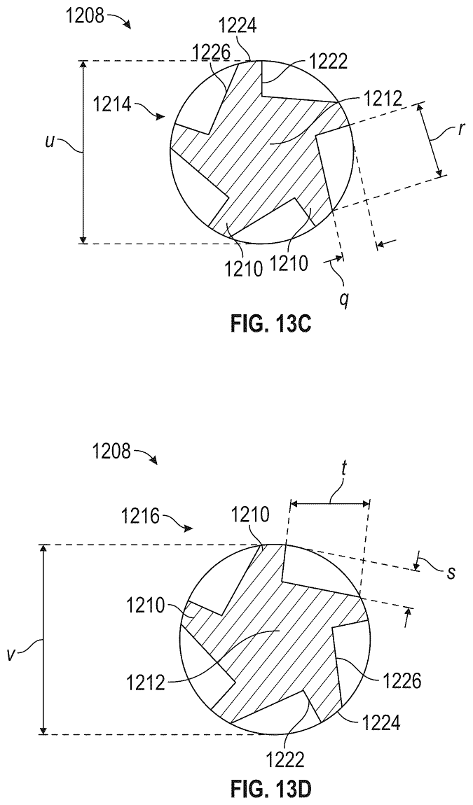

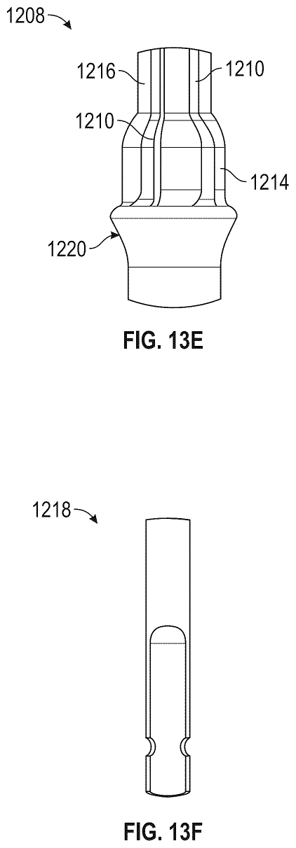

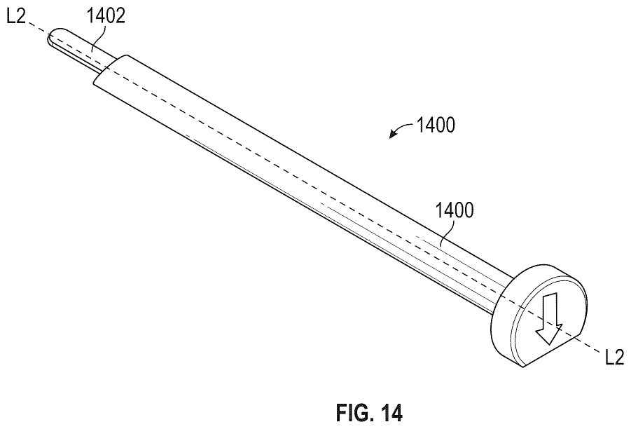

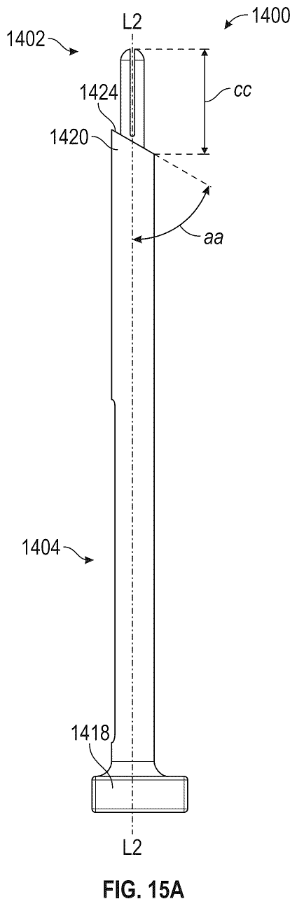

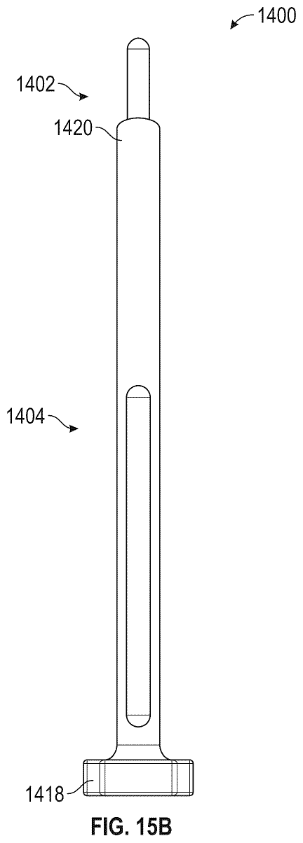

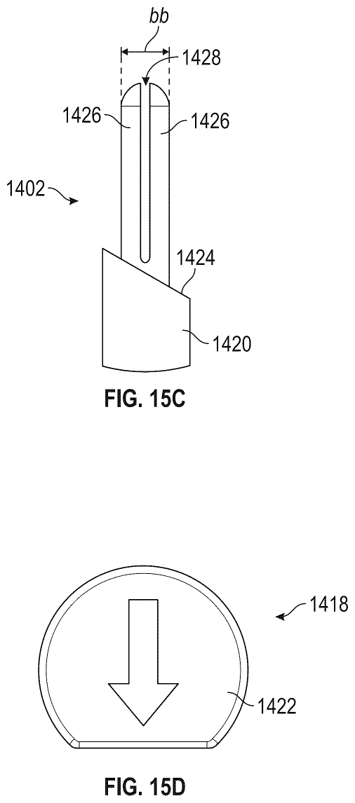

FIGS. 12-13F illustrate an exemplary reamer apparatus configured for use with the exemplary orthopedic inserts disclosed herein and/or other orthopedic inserts. FIGS. 14-15D illustrate an exemplary inserter apparatus configured for use with the exemplary orthopedic inserts disclosed herein and/or other orthopedic inserts.

### Exemplary Orthopedic Inserts

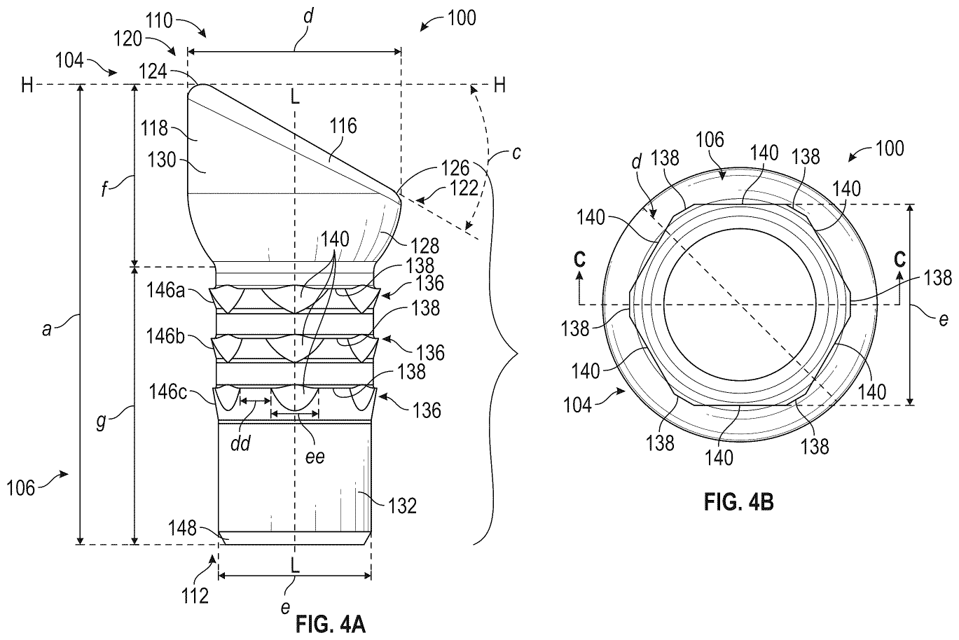

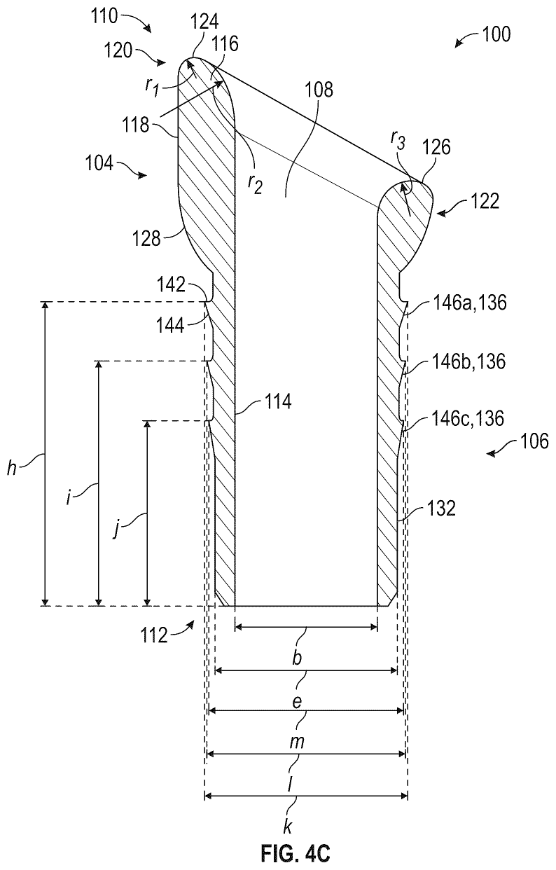

Turing to FIGS. 3A-8, an exemplary orthopedic insert 100 in accordance with the present disclosure is shown and described. As can be seen therein, the orthopedic insert 100 can include an insert body 102 that has a head portion 104 and a shaft portion 106 extending from the head portion along a longitudinal axis L-L of the insert body 102. The head portion 104 can define a first end 110 of the insert body 102 and the shaft portion 106 can define a second end 112 of the insert body 102. As shown in FIG. 4A, the insert body can have an overall length a (as measured along the longitudinal axis). In some examples, the length a is in a range of 5 mm to 50 mm, such as 5 mm to 40 mm, 5 mm to 30 mm, 5 mm to 20 mm, 5 mm to 15 mm, 10 mm to 50 mm, 10 mm to 40 mm, 10 mm to 30 mm, 10 mm to 20 mm, 10 mm to 15 mm, etc. In some examples, the length a is in a range of 10.1 mm to 14.1 mm.

The insert body 102 can include a bore 108 extending therethrough along a longitudinal axis L-L between the second end 112 and a rim (also referred to as a rim portion) 116 of the head portion 104 (disposed at the first end 110 of the insert body 102). In some examples, the bore 108 can be defined by a continuous smooth interior surface or wall 114. In some examples, the bore 108 can have a substantially continuous or constant diameter over a length of the insert body 102. In some examples, the bore 108 that can have a substantially continuous or constant diameter from the second end 112 of the insert body to the rim 116. For example, as shown in the cross-sectional view of FIG. 4C, the bore 108 can have a constant diameter b. In some examples, the diameter b can be in a range of 1 mm to 10 mm, such as 2 mm to 10 mm, 2 mm to 8 mm, 2 mm to 6 mm, 2 mm to 5 mm, etc. In some examples, the diameter b can be in a range of 1.9 mm to 4.6 mm. In other examples, the bore can have varying diameter over a length of the bore. For example, the bore can have tapered diameter, such as a constantly increasing or decreasing diameter from the second end of the insert body toward the first end of the insert body.

As noted above, in some examples, the head portion 104 can include the rim 116, and an interior portion of the rim 116 can circumferentially define a first opening of the bore 108. In some examples, the rim 116 can be a sloped rim that is disposed at or defines an angle c relative to a plane H-H that is perpendicular to the axis L-L and tangent to the apex 124 of the rim (FIG. 4A). The angle c can be the complement to the angle of the rim measured relative to the longitudinal axis L-L of the insert body 102. For example, the sloped rim 116 can be disposed at an angle c in a range of 5° to 60°, such as 5° to 45°, 10° to 60°, 10° to 45°, 20° to 60°, 20° to 45°, 25° to 35°, etc., relative to the longitudinal axis L-L of the insert body 102. In another example, the sloped rim 116 can be disposed at a 30° angle relative to the longitudinal axis L-L of the insert body 102. In certain examples, the angle of the rim 116 can be selected to approximate the angle of the outer surface of the bone at the location where the insert is to be implanted, such as, for example, the lateral condyle of the femur.

The sloped rim 116 can give the head portion 104 an overall sloped shape. Thus, in examples including a sloped rim, the head portion 104 can include an upper or extended portion 120 and a lower or base portion 122 at a (proximal) face of the insert body 102. The upper portion 120 can be farther from the second end 112 of the insert body 102 along the longitudinal axis L-L than the lower portion 122. A highest point or center point of the upper portion 120 can form an apex 124 of the sloped rim 116, and a point 126 diametrically opposing the apex 124 can form a lowest or base point 126 of the sloped rim 116.

As shown in FIG. 4C, the curved surface of the rim 116 can have a first radius r₁ at the apex 124 that extends to an exterior surface 118 of the head portion 104. In some examples, the first radius r₁ is in a range of 0.1 mm to 0.8 mm, such as 0.2 mm to 0.5 mm, 0.3 mm to 0.5 mm, or 0.3 mm to 0.8 mm, etc. In some examples, the first radius r₁ is 0.4 mm. Further, the curved surface of the rim 116 can have a second radius r₂ at the apex 124 that extends to the interior wall 114. In some examples, the second radius r₂ is greater than the first radius r₁. In some examples, the radius r₂ is in a range of 0.4 mm to 4 mm, such as 0.5 mm to 3 mm, 0.8 mm to 2 mm, etc. In some examples, the radius r₂ is 2 mm at the location of the apex 124 on the upper portion 120 of the rim. In some examples, the second radius r₂ can vary around the circumference of the rim 116. For example, in certain examples the radius r₂ can increase from a minimum value (also referred to as a third radius r₃) at the point 126 of the lower portion 122 of the rim to a maximum value at the apex 124 of the upper portion 120 of the rim. In a particular example the radius r₂ can increase from 0.8 mm at the lowest point 126 to 2 mm at the apex 124. Stated differently, the curved surface of the rim 116 can have a third radius r₃ between the lowest point 126 and the interior wall 114. In some examples, the third radius r₃ is greater than the first radius r₁ and is less than or equal to the second radius r₂ at the apex 124. In some examples, the third radius r₃ is in a range of 0.3 to 3 mm, such as 0.5 to 2.5 mm, 0.8 mm to 2 mm, etc. In some examples, the third radius r₃ is 0.8 mm at the location of the lowest point 126.

The head portion 104 can further include a base portion 128 that is attached to or integral with the shaft portion 106. The base portion 128 can curve outwardly from or relative to the shaft 106 such that a width or diameter d of the head portion 104 is greater than a width or diameter e of the shaft 106 (FIG. 4A). In some examples, the width d is in a range of 2 mm to 12 mm, such as 2 mm to 10 mm, 3 mm to 10 mm, 4 mm to 10 mm, 2 mm to 8 mm, 3 mm to 8 mm, 4 mm to 8 mm, 4.0 mm to 6.0 mm, etc. In some examples, the width e is in a range of 1 mm to 10 mm, 1 mm to 8 mm, 1 mm to 6 mm, 2 mm to 10 mm, 2 mm to 8 mm, 2 mm to 6 mm, 2.5 mm to 3.5 mm, etc. In one specific example, the width d is 4.65 mm and the width e is 2.90 mm. In another specific example, the width d is 5.15 mm and the width e is 3.40 mm. In yet another specific example, the width d is 5.75 mm and the width e is 4.10 mm. In still another specific example, the width d is 7.15 mm and the width e is 5.40 mm.

The base portion 128 extends between the shaft 106 to the lowest point 126 of the sloped rim 116 and a lower end or boundary of a partially cylindrical wall 130 of the head portion 104. The wall 130 is parallel to the longitudinal axis L-L and extends between the base portion 128 and the sloped rim 116 (e.g., a portion of the sloped rim 116 circumferentially offset from the lowest point 126).

The head can have an overall length f, which can be less than a length g of the shaft 106. In some examples, the length f is in a range of 2 mm to 10 mm, such as 2 mm to 8 mm, 2 mm to 6 mm, 3 mm to 10 mm, 3 mm to 8 mm, 3 mm to 6 mm, 4 mm to 5 mm, etc. In some examples, the length g is in a range of 2 mm to 15 mm, such as 2 mm to 10 mm, 4 mm to 15 mm, 4 mm to 10 mm, 4 mm to 8 mm, 6 mm to 15 mm, 6 mm to 10 mm, 6 mm to 8 mm, etc. In one specific example, where the length a of the insert body 102 is 10.1 mm, the length f is 4.1 mm and the length g is 6.0 mm. In another specific example, where the length a of the insert body 102 is 11.3 mm, the length f is 4.5 mm and the length g is 6.8 mm. In yet another specific example, where the length a of the insert body 102 is 12.4 mm, the length f is 4.9 mm and the length g is 7.5 mm. In still another specific example, where the length a of the insert body 102 is 14.1 mm, the length f is 6.1 mm and the length g is 8.0 mm.

In other examples, the head portion can have other configurations (see, for example, the exemplary orthopedic inserts 800, 900, 1000 shown in FIGS. 9-10 and discussed further below).

Referring again to FIG. 4A, as discussed above the shaft portion 106 extends from the head portion 104 along the longitudinal axis L-L of the insert body 102, and the shaft portion can have the width e and the length g. In some examples, the shaft 106 can be generally cylindrical in shape and include an exterior surface 132. In some examples, the shaft 106 includes a chamfer 148 at the second end 112 of the insert body. The chamfer 148 can surround a second opening to the bore 108 at the second end 112 of the insert body (opposing the first opening of the bore 108 defined by the interior of the rim 116). In some examples, a length of chamfer is in a range of 0.1 to 0.3 mm. In some examples, the chamfer 148 defines or is disposed at an angle in a range of 30° to 80°, such as 35° to 75°, 40° to 70°, etc. relative to the longitudinal axis L-L. In one specific example, the chamfer 148 defines or is disposed at a 60° angle relative to the longitudinal axis L-L. In other examples the second end 112 of the insert body can be beveled, rounded or filleted, and/or can comprise a 90° corner between the surface 132 and the distal surface of the second end 112.

In some examples, the insert body 102 further includes a plurality of annular ribs 136 extending outwardly from the exterior surface 132 of the shaft portion 106 that are spaced apart from each other along the longitudinal axis L-L. In some examples, the annular ribs 136 can be formed, molded, or machined to include a plurality of projections 138. For example, the annular ribs 136 can include a plurality of recesses 140, which can be configured as facets or cutaways (e.g., curved or flat surfaces). The recesses 140 can be circumferentially arranged on each annular rib, and the non-flat portions disposed between adjacent recesses 140 can form the projections 138. In the present example, the annular ribs 136 each include six recesses 140 (FIG. 4B). In other examples, the annular ribs can include more or fewer facets or recesses, and the insert body can have a different configuration corresponding to the number of facets or cutaways. The recesses 140 can extend longitudinally all the way through the ribs 136 (as in, for example, the uppermost row 146a in FIG. 4A), or part way through the ribs (as in, for example, the lowermost row 146c).

As best seen in FIG. 4C, in some examples, each annular rib 136 includes a first (top) surface 142 transverse to the longitudinal axis L-L and a second (bottom) surface 144 angled relative to the longitudinal axis L-L, where the first surface 142 is oriented toward the first end 110 of the insert body 102 and the second surface 144 is oriented toward the second end 112 of the insert body 102. Thus, in such examples, the projections 138 formed on the ribs 136 can be transverse or substantially transverse relative to the longitudinal axis L-L of the insert body 102 (e.g., the projections can extend radially outwardly form the exterior surface 132). Further, an intersection between the first surface 142 and the second surface 144 at each of the projections 138 can form an exterior or radially outward edge of the projection. The projections 138 may also be referred to as barbs, extensions, spurs, protrusions, etc. In other examples, the projections can have other configurations, such as spikes, hooks, points, or bumps. In other examples, the plurality of projections can include a combination of the foregoing projection configurations. In some examples, the insert body can exclude annular ribs, and the projections can be molded or integral with, or attached to the exterior surface of the shaft.

For any of the foregoing exemplary configurations of the projections, the projections 138 can be arranged on the shaft 106 in a plurality of rows 146 (formed by an annular ribs 136) spaced apart along the longitudinal axis L-L of the insert body 102. Specifically, the present example includes three rows 146a, 146b, 146c of projections 138 spaced apart from each other on the exterior surface 132 of the shaft 106 along the longitudinal axis L-L. In some examples, the plurality of rows can be evenly spaced (i.e., having a gap of equal distance between adjacent ones of the rows). In some examples, the plurality of rows can be unevenly spaced (i.e., having one or more gaps that have a distance different relative to one or more other gaps between adjacent ones of the rows).

As can be seen in FIG. 4C, the projections 138 in the row 146a are a distance h from the second end 112 of the insert body 102, the projections 138 in the row 146b are a distance i from the second end 112 of the insert body 102, and the projections 138 the row 146c are a distance j from the second end 112 of the insert body 102. In some examples, the distance h is in a range of 3 mm to 12 mm, such as 3 mm to 10 mm, 3 mm to 8 mm, 4 mm to 12 mm, 4 mm to 10 mm, 5 mm to 8 mm, etc. In some examples, the distance i is in a range of 2 mm to 10 mm, such as 2 mm to 8 mm, 2 mm to 6 mm, 3 mm to 10 mm, 3 mm to 8 mm, 3 mm to 6 mm, 4 mm to 6 mm, etc. In some examples, the distance j is in a range of 1 mm to 8 mm, such as 1 mm to 6 mm, 1 mm to 4 mm, 2 mm to 8 mm, 2 mm to 6 mm, 2 mm to 5 mm, etc. In one specific example, the distance h is 5.40 mm, the distance i is 4.05 mm, and the distance j is 2.70 mm. In another example, the distance h is 6.20 mm, the distance i is 4.85 mm, and the distance j is 3.50 mm. In yet another example, the distance h is 6.90 mm, the distance i is 5.55 mm, and the distance j is 4.20 mm. In still another example, the distance h is 7.40 mm, the distance i is 6.05 mm, and the distance j is 4.70 mm.

Also shown in FIG. 4C, the curved, radially outward edges of the projections 138 can have a diameter. Specifically, in the example of FIG. 4C, the projections 138 in row 146a have a diameter k, the projections 138 in row 146b have a diameter l, and the projections 138 in row 146c can have a diameter m. In some examples, the diameter k is greater than the diameter l, and the diameter l is greater than the diameter m. Stated differently, the projections 138 in the row 146a can extend radially outward from the exterior surface 132 by a greater distance than the projections of row 146b and the projections of row 146c. In some examples, each of the diameters k, l, m is in a range of 2 mm to 7 mm, such as 2.5 mm to 6.5 mm, 2 mm to 6 mm, 2 mm to 5 mm, etc., with a difference in projection diameter between adjacent rows 146a, 146b, 146c being in a range of 0.01 mm to 0.3 mm, such as 0.05 mm to 0.2 mm, 0.05 to 0.1 mm, etc. In one specific example, the diameter k is 3.46 mm, the diameter l is 3.40 mm, and the diameter m is 3.30 mm. In another example, the diameter k is 4.00 mm, the diameter l is 3.90 mm, and the diameter m is 3.80 mm. In yet another example, the diameter k is 4.60 mm, the diameter l is 4.50 mm, and the diameter m is 4.40 mm. In still another example, the diameter k is 6.00 mm, the diameter l is 5.90 mm, and the diameter m is 5.80 mm.

The projections 138 in each of the rows can also have a height, which can be measured relative to the exterior surface of the shaft 106. In some examples, a height of the projections 138 in the row 146c is in a range of 0.1 mm to 0.6 mm, such as 0.2 to 0.5 mm, 0.3 mm to 0.4 mm, etc. In some examples, a height of the projections 138 in the row 146b is in a range of 0.2 mm to 0.7 mm, such as 0.3 to 0.6 mm, 0.4 mm to 0.5 mm, etc. In some examples, a height of the projections 138 in the row 146a is in a range of 0.3 mm to 0.8 mm, such as 0.4 to 0.7 mm, 0.5 mm to 0.6 mm, etc.

Thus, in the present examples the diameter of the projections 138 and/or the height of the projections 138 above the surface 132 increases in a direction along the longitudinal axis L-L from the second end 112 toward the first end 110 of the insert body 102. Stated differently, the height of the projections 138 in the rows 146c, 146b, and 146a increases in the direction along the longitudinal axis L-L from the second end 112 toward the first end 110 of the insert body 102. In some examples, a length dd of the exterior or radially outward or perimeter edge of the projections 138 (e.g., measured along the exterior edge at the intersection of the first and second surfaces 142, 144, which, in some examples, is a curved edge and, in some examples, is a linear or non-curved edge) decreases in the direction along the longitudinal axis L-L from the second end 112 toward the first end 110 of the insert body 102. Thus, in certain examples the length dd of the perimeter edges of the projections 138 in row 146c is greater than that of the projections 138 in rows 146b and 146a, and the length of the perimeter edges of the projections 138 in row 146b is greater than that of the projections 138 in row 146a. In some examples, a maximum width ee and/or a surface area of the recesses 140 can increase in the direction along the longitudinal axis L-L from the second end 112 toward the first end 110 of the insert body 102 with the recesses in the row 146c having the smallest width dimensions and the recesses in the row 146a having the largest width dimensions. In other examples, the projections in any or all of the rows can end in points.

In other examples, the insert body can include more or fewer rows of projections. In still other examples, the projections can have different arrangements, such as, for example, be clustered or evenly dispersed over the exterior surface of the shaft rather than being arranged in rows. Further, in other exemplary orthopedic inserts the shaft can include an exterior surface without any annular ribs and/or projections (e.g., a smooth exterior surface or a surface having a high-friction coating or treatment).

In some examples, the orthopedic inserts can be manufactured from one or more biocompatible materials, such as, for example, titanium, stainless steel (e.g., ASTM 316, 316L, or other chromium-nickel-molybdenum stainless steels), and/or polyether ether ketone (PEEK). In some examples, the orthopedic inserts can be manufactured from one or more bioresorbable materials, such as, for example, Poly (D,L-lactide-co-glycolide), poly(E-caprolactone), and/or poly(butylene succinate). In examples where the orthopedic inserts are manufactured from bioresorbable materials, the orthopedic insert can be configured to dissolve over a period of time, for example, over a period of six to nine months, giving the body sufficient time to form fibrous tissue to stabilize the treated joint.

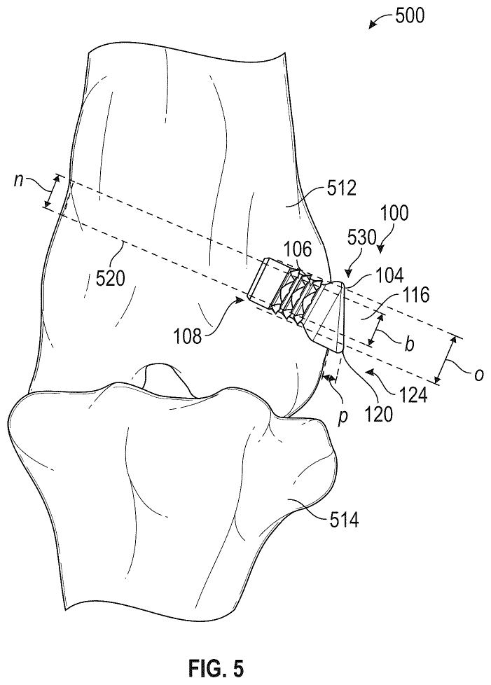

Turning to FIG. 5, an exemplary insertion configuration or state 500 for the orthopedic insert 100 is illustrated. Specifically, FIG. 5 shows a bone tunnel 520 through a first bone 512 (e.g., a femur) of a canine stifle joint for treatment or reconstruction via a lateral suture technique. A second bone 514 (e.g., a tibia) of the stifle joint is disposed below the first bone 512. In order to stabilize or protect the bone at or proximate the exit portion 530 of the bone tunnel 520, the orthopedic insert 100 can be (at least partially) seated (e.g., inserted) within the exit portion 530 (e.g., the lateral opening of the bone tunnel in FIG. 5). In some examples, the bone tunnel 520 can be sized such that there is an interference fit between orthopedic insert 100 and the walls of the bone tunnel 520.

As can be seen in FIG. 5, the bone tunnel can have a width or diameter n, which may be sized dependent upon the anatomy of the patient (human or animal), the condition of the knee, and/or other factors. In some examples, the orthopedic insert 100 may be selected from a set or a kit of orthopedic inserts of varying sizes or can be manufactured (e.g., 3D printed) to a specified size dependent upon the anatomy of the patient (human or animal), the condition of the knee, and/or other factors (such as, for example, age of the patient, frequency of strenuous activities performed by the patient, etc.). In some examples, the bone tunnel 520 can be formed to have a specified diameter for treatment, and an orthopedic insert 100 can be selected or manufactured so that the diameter b of the bore 108 is equal to or substantially equal to the diameter n of the bone tunnel 520. In other examples, an orthopedic insert of a specified size can be selected for treatment, and the bone tunnel 520 can be formed to match diameter b the bore 108 the orthopedic insert 100. Stated differently, in instances where a specified bone tunnel diameter is indicated (e.g., due to anatomical constraints), the bone tunnel diameter can determine the size of the orthopedic insert selected. In instances where a specified orthopedic insert size is indicated, the selected orthopedic insert size can be used determine a diameter of the formed bone tunnel.

In the foregoing examples, the inside wall of the bone tunnel 520 and the bore of the insert body 102 can define a continuous lumen of constant or substantially constant diameter. This can prevent a step or sudden change in diameter at the interface between the bone tunnel 520 and the orthopedic insert which could abrade a suture moving within the bone tunnel during flexion and extension of the joint.

Also illustrated in FIG. 5, the exit portion 530 of the bone tunnel 520 can be formed (for example, reamed using an apparatus, such as, for example, the reamer apparatus shown in FIGS. 12-13F and discussed further below) to a diameter or width o that is greater than the diameter n in order to accommodate seating of the orthopedic insert 100 therein. In some examples, the diameter o is approximately equal to the outer diameter e (FIG. 4C) of the shaft portion 106 of the insert body 102. In some examples, the diameter o can be slightly larger or smaller than the diameter e of the shaft portion 106 of the insert body 102. In some examples, such as in the example shown in FIG. 7, the exit opening of the exit portion of the bone tunnel can be formed to have a greater diameter than the diameter o in order to accommodate seating of the head portion 104 therein. In certain examples, the bone tunnel can comprise a countersink at, for example, the lateral exit that is configured to receive the insert and sized to result in an interference fit between the bone and the insert to retain the insert in the bone tunnel.

In the foregoing examples, the exit portion 530 of the bone tunnel 520 can be formed such that at least the shaft portion 106 of the insert body can be seated therein, and the projections 138 extending from the shaft can engage with or be anchored into bone on the interior surface of the bone tunnel 520. The engagement between and/or anchoring of the projections 138 into the bone on the interior surface of the bone tunnel 520 can prevent or limit movement of the orthopedic insert 500 relative to the bone tunnel 520.

As discussed above, in some examples, a height of the projections 138 in the respective rows (such as, for example, the rows 146a, 146b, 146c) decreases in a direction from the first end 110 to the second end 112 of the insert body. Thus, in the present example, the projections 138 in row 146a can have a greater height than the projections 138 in the row 146b, and the projections 138 in the row 146b can have a greater height than the projections 138 in the row 146c, which can limit insertion or movement of the orthopedic insert 100 further into the bone tunnel 520 (for example, in a lateral to medial direction) past a desired depth. For example, the projections 138 can limit the orthopedic insert 100 from being inserted into the bone tunnel 520 past the exit portion 530. In some examples, the engagement between the projections 138 and the bone on the interior surface of the bone tunnel 520 can limit or prevent movement or withdrawal of the orthopedic insert 100 from of the bone tunnel 520 (for example, in a medial to lateral direction).

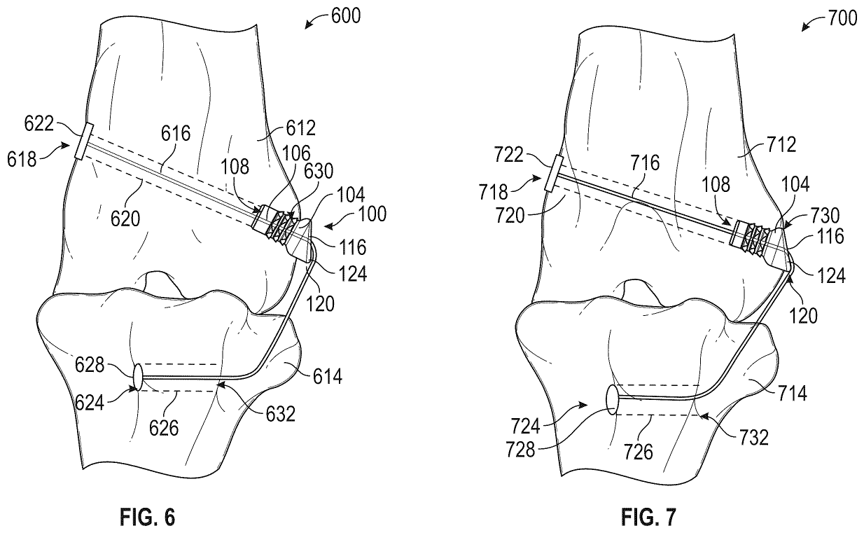

In some examples, as illustrated in FIGS. 5 and 6, the orthopedic insert 100 can be partially inserted into a bone tunnel such that a proximal face (for example, the rim 116) of the head portion 104 is offset relative to an exterior surface of the first bone. For example, the apex 124 of the upper or extended portion 120 of the head can be offset from the surface of the first bone 512 by an offset distance p. In some examples, the offset distance p is in a range of 0.5 mm to 10 mm, such as 1 mm to 8 mm, 2 mm to 6 mm, 3 mm to 10 mm, 3 mm to 8 mm, 3 mm to 6 mm, 4 mm to 5 mm, etc. In some examples, as in FIG. 7, the orthopedic insert 100 can be fully inserted into a bone tunnel such that a proximal face (for example, the rim 116) of the head portion 104 is flush or substantially flush with an exterior surface of the first bone. For example, the rim 116 can be offset less than 0.5 mm from the surface of the first bone.

After seating, insertion, press fitting, or interference fitting of the orthopedic insert into a bone tunnel in a first bone, a suture can be threaded through the bone tunnel and the bore of the orthopedic insert. For example, FIG. 6 shows an exemplary suture configuration 600 through a first bone 612 (e.g., a femur) and a second bone 614 (e.g., a tibia) of a canine stifle joint after treatment or reconstruction via a lateral suture technique including utilization of the orthopedic insert 100, where the orthopedic insert 100 is partially inserted into a femoral bone tunnel 620. Although the bone tunnel 620 is shown as having a similar width to the orthopedic insert 100, it can have a width that corresponds to the width of the bore 108 of the orthopedic insert (as discussed above with reference to FIG. 5). In some examples, a first end of a suture 616 can be fixed or secured at an entrance 618 to the femoral bone tunnel 620 utilizing a first anchoring device 622, such as a button-type anchor (FIG. 2B) or other anchor. In some examples, a second (opposing) end of the suture 616 can be fixed or secured at an exit 624 of a tibial bone tunnel 626 utilizing a second anchoring device 628, such as an interference screw (FIG. 2C) or other anchor.

As can be seen in FIG. 6, at an exit portion 630 of the femoral bone tunnel 620, a central portion of the suture 616 extends from the bone tunnel 620 through the bore 108 of the orthopedic insert 100, thereby limiting or preventing contact between the suture 616 and the bone at the exit portion 630. Specifically, the suture 616 extends over the rim 116 of the head portion 104 at the extended or upper portion 120, which can guide the suture away from the bone and surrounding soft tissue. The suture can and wrap or turn at an angle to extend over the surface of a lateral aspect of the femur 612 toward the tibial bone tunnel 626. In such examples, the bone at the exit portion 630 can be protected from wear by the tensioned suture 616. Thus, the tension on the suture 616 and stability of the treated knee joint can be better maintained overtime relative to other lateral suture treatments.

Further, because of the offset of the rim 116 of the head portion 104 of the orthopedic insert 100 from the surface of the first bone 612, the suture 616 can additionally have limited or decreased contact with soft tissue proximate the exit portion 630 of the femoral bone tunnel 620 relative to existing lateral suture treatments. Thus, when partially inserted into a bone tunnel, the orthopedic insert 100 can reduce pain resulting from the contact between or compression of the soft tissue by the suture.

In some examples, full insertion of all or substantially all of the orthopedic insert into the bone may be indicated, for example, due to the anatomy of a particular patient. For example, FIG. 7 shows an exemplary suture configuration 700 through a first bone 712 (e.g., a femur) and a second bone 714 (e.g., a tibia) of a stifle joint after treatment or reconstruction via a lateral suture technique including utilization of the orthopedic insert 100, where the orthopedic insert 100 is fully inserted into a femoral bone tunnel 720. Although the bone tunnel 720 is shown as having a similar width to the orthopedic insert 100, it can have a width that corresponds to the width of the bore 108 of the orthopedic insert (as discussed above with reference to FIG. 5). In some examples, a first end of a suture 716 can be fixed or secured at an entrance 718 to the femoral bone tunnel 720 (e.g., on the medial aspect) utilizing a first anchoring device 722, such as a button-type anchor (FIG. 2B) or other anchor. In some examples, a second (opposing) end of the suture 716 can be fixed or secured at an exit 724 (e.g., a medial opening in FIG. 7) of a tibial bone tunnel 726 utilizing a second anchoring device 728, such as an interference screw (FIG. 2C) or other anchor.

As can be seen in FIG. 7, at an exit portion 730 (e.g., the lateral opening in FIG. 7) of the femoral bone tunnel 720, a central portion of the suture 716 extends from the bone tunnel 720 through the bore 108 of the orthopedic insert 100, thereby preventing or limiting contact between the suture 716 and the bone at the exit portion 730. Specifically, the suture 716 extends over the rim 116 of the head portion 104 at the extended or upper portion 120 and wraps or turns at an angle to extend over the surface of a lateral aspect of the femur 712 toward the tibial bone tunnel 726. In such examples, the bone at the exit portion 730 can be protected from wear by the tensioned suture 716. Thus, the tension on the suture 716 and stability of the treated stifle joint can be better maintained overtime relative to existing lateral suture treatments.

As can be seen in FIGS. 6 and 7, the angled proximal face at the rim 116 of the head portion 104 (formed by the upper or extended portion 120 and the lower or base portion 122) can be configured to match or align with the curvature of the first bone 612, 712 and limit protrusion of the orthopedic insert 100 into the patient's anatomy. Further, the spherical or curved shape of the head portion 104 (formed by the curved base 128) can provide improved stress distribution to the first bone 612, 712 during loading on the treated joint.

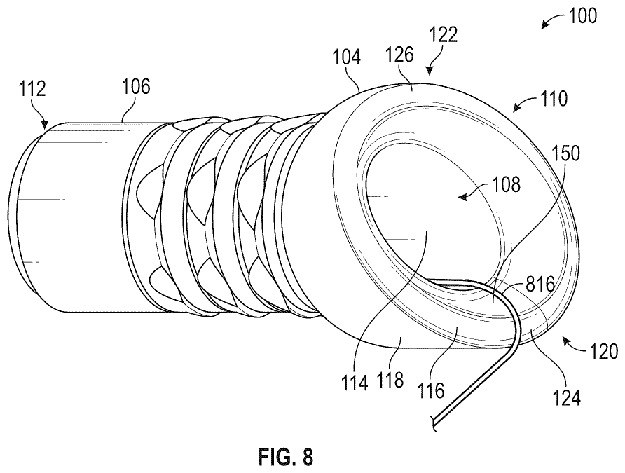

FIG. 8 shows a detailed view of the orthopedic insert 100 and a suture 816 extending through the bore 108. The suture 816 (as well as one or more of the other sutures disclosed herein) can be made of a high-tensile strength material. In some examples, the suture 816 can be made from an absorbable material, such as Monocryl®, Vicryl®, polydioxanone (PDS), or collagen. The suture materials can be in the form of a monofilament or multifilament (e.g., braided) thread. As can be seen in FIG. 8, the curved surface of the rim 116 extending between the interior wall 114 of the bore 108 and the upper or extended portion 120 of the head portion 104 forms a bearing surface 150 configured to support, receive, or contact the suture 816. The bearing surface 150 can be a polished surface or low friction surface that is free or substantially free of sharp edges. The bearing surface 150 can thereby facilitate low friction movement of the suture 816 over the bearing surface 150 and can limit wear and/or rupture of the suture overtime.

As discussed above, in other examples, an orthopedic insert can include a head portion with a different configuration. For example, FIGS. 9-11 show exemplary orthopedic inserts 900, 1000, 1100 that can be similar to the orthopedic insert 100 and can be used in a similar manner (for example, in a lateral suture technique procedure), but have a differently configured head. Specifically, in one example, the orthopedic insert 900 of FIG. 9 includes a shaft portion 906 that can have features similar to the shaft portion 106, and a head portion 904 that can have a similar configuration to the curved base 128 but lacks any extended/upper portion and recessed/lower portion. In other words, the head portion 904 can include a curved wall that curves radially outwardly from the shaft portion 906, and a proximal face or rim of the head portion 904 can be perpendicular or substantially perpendicular to a longitudinal axis of the orthopedic insert 900.

In another example, the orthopedic insert 1000 of FIG. 10 includes a shaft portion 1006 that can have features similar to the shaft portion 106, and a head portion 1004 that has an overall a frustoconical shape. Similar to the head portion 904, a proximal face or rim of the head portion 1004 can be perpendicular or substantially perpendicular to a longitudinal axis of the orthopedic insert 1000.

In yet another example, the orthopedic insert 1100 of FIG. 11 includes a shaft portion 1106 that can have features similar to the shaft portion 106, and a head portion 1104 that has an overall a tubular shape. In other words, the exterior surface of the head portion 1104 can be coextensive with the exterior surface of the shaft portion 1106, and a proximal face or rim of the head portion 1104 can be perpendicular or substantially perpendicular to a longitudinal axis of the orthopedic insert 1100.

In other examples, the foregoing head portions 904, 1004, 1104 can include one or more features of the head portion 104. For example, the foregoing head portions 904, 1004, 1104 can include a proximal face or rim that is angled relative to a longitudinal axis of the orthopedic insert.

Each of the orthopedic inserts 900, 1000, and 1100 can also include a plurality of ribs longitudinally spaced apart along the shaft portion. In FIGS. 9-11 the ribs are continuous in the circumferential direction, but the ribs can also be machined to include recesses and projections as described above with reference to the orthopedic insert 100.

### Exemplary Inserter and Reamer Apparatus

The foregoing orthopedic inserts can be a component of or can be used in combination with an orthopedic insert system. For example, FIGS. 12-13D illustrate an exemplary reamer apparatus 1200 and FIGS. 14-15F illustrate an exemplary inserter apparatus 1400, which can be utilized to prepare the bone and implant the orthopedic inserts disclosed herein. In some examples, the exemplary orthopedic inserts disclosed herein can be utilized with other reamer apparatus and/or other inserter apparatus. In some examples, the exemplary reamer apparatus 1200 can be utilized with other orthopedic inserts.

As can be seen in FIGS. 12-13D, the reamer apparatus 1200 incudes a shank portion 1204 (also referred to as a shaft) and a cutting head portion 1202 extending from the the shank portion 1204 along a longitudinal axis L1-L1 of the reamer apparatus. In some examples, the shank portion 1204 can be a handle portion for operation of the reamer apparatus 1200. In some examples, the shank portion 1204 can be configured for attachment to a motorized or robotic device for operation of the reamer apparatus 1200, such as any of a variety of surgical reamer drivers or drills. For example, the proximal end portion 1218 of the shaft 1204 can include a coupling configured for attachment to a driver. In some examples, the shank portion 1204 further includes an opposing distal end portion 1220 configured be coupled to, attached to, or to be integral with the cutting head portion 1202.

The cutting head portion 1202 can include a guide nose or pilot 1206 and a cutter 1208. In some examples, the cutter 1208 can include a plurality of cutting edges or blades 1210 and flutes circumferentially arranged on and extending from a core body 1212 (FIGS. 13C and 13D). The core body 1212 can be axially aligned with the pilot 1206 along the longitudinal axis L1-L1. In some examples, the flutes and/or blades 1210 are parallel to the longitudinal axis L1-L1. In other examples, the flutes and/or blades can be angled relative to the longitudinal axis L1-L1.

In some examples, the cutter 1208 incudes a proximal portion 1214 and a distal portion 1216. As can be seen in FIGS. 13C and 13D, the proximal portion 1214 can have a diameter u and the distal portion 1216 can have a diameter v that is less than the diameter u. The proximal portion 1214 and the distal portion 1216 can each have five blades 1210, and the blades can be continuous from the proximal portion to the distal portion with a diameter reduction from the diameter u to the diameter v in a transition region at the distal end of the proximal portion 1214. As can be seen in FIG. 13B, the pilot 1206 can have a diameter w, which can be smaller than each of the diameters v and u.

In the illustration configuration, the cutter 1208 can be shaped for rotation clockwise direction in FIGS. 13C and 13D. The blades 1210 can thus comprise a leading surface 1222, a radially outer surface 1224, and a trailing surface 1226. In the proximal portion 1214 the leading surfaces 1222 of the blades 1210 can have a height q, and the trailing surfaces 1226 can have a length r. On the distal portion 1216, the leading surfaces 1222 can have a height s and the trailing surfaces 1226 can have a length t, which can be less than the corresponding dimensions q and r on the proximal portion 1214. In other examples, the height q can be equal to the height s, while the length r is greater than length t.

In some examples, the height q is in a range of 0.2 mm to 2 mm, such as 0.3 mm to 1.5 mm, 0.5 to 1 mm, etc. In some examples, the width r is in a range of 0.5 to 5 mm, such as 1 mm to 4 mm, 1.5 mm to 3 mm, etc. In some examples, the diameter u is in a range of 2 mm to 11 mm, such as 3 mm to 10 mm, 4.5 mm to 7.5 mm, etc. In some examples, the height s is in a range of 0.2 mm to 1.2 mm, such as 0.3 mm to 0.8 mm, 0.5 mm to 0.6 mm, etc. In some examples, the width t is in a range 0.6 mm to 5 mm, such as 0.8 mm to 3 mm, 1.2 mm to 2.3 mm, etc. In some examples, the diameter v is in a range of 1.5 mm to 8 mm, such as 2 mm to 6.5 mm, 2.9 mm to 5.5 mm, etc. In some examples, the diameter w which can be in a range of 0.9 mm to 7 mm, such as 1.5 mm to 6 mm, 1.8 mm to 4.5 mm, etc.

Thus, in some examples, the cutting head portion 1202 can have an overall shape and size where the pilot 1206 is a narrowest portion of the cutting head portion 1202 and is configured for insertion into an exit portion of a pre-formed bone tunnel (for example, the exit portion 530 of the bone tunnel 520 illustrated in FIG. 5). Further, the distal portion 1216 of the cutter 1208 can have an intermediate diameter relative to the pilot 1206 and the proximal portion 1214 of the cutter 1208.

When the cutter 1208 is rotated within the bone tunnel, the distal portion 1216 of the cutter 1208 can be configured to widen the pre-formed bone tunnel to a first diameter (e.g., the diameter v), while the proximal portion 1214 of the cutter 1208 can be configured to widen the pre-formed bone tunnel to a second diameter (e.g., the diameter u) that is greater than the first diameter. The configuration of the cutter 1208 can create a counterbore (also referred to as a countersink) in the opening of the bone tunnel, resulting in the exit portion of the bone tunnel proximate the opening, which is reamed by the proximal portion 1214 of the cutter, having a greater diameter than the portion of the bone tunnel reamed by the distal portion 1216. Thus, the resulting shape of the exit portion of the bone tunnel can generally correspond to a shape of the shaft and head portions of the orthopedic insert (such as, for example, a shape of the shaft portion 106 and the head portion 104 of the orthopedic insert 100 in FIGS. 3A-8). Accordingly, in some examples, a reamer apparatus can be selected or manufactured to have dimensions corresponding to a diameter of the pre-formed bone tunnel and/or a size of a selected orthopedic insert.

As discussed above, in some examples or conditions, an orthopedic insert can be fully inserted into an opening of a bone tunnel such that a proximal face of the orthopedic insert is flush with an exterior surface of the bone (FIG. 7). In other examples or conditions, an orthopedic insert can be partially inserted into an opening of a bone tunnel such that a proximal face of the orthopedic insert is offset from an exterior surface of the bone (FIGS. 5 and 6). A depth of insertion of an orthopedic insert can be controlled at least in part by the forming or sizing of the opening of the bone tunnel with the reamer apparatus. Thus, in some examples, a reamer apparatus can be further selected or manufactured to have dimensions corresponding to a desired depth of insertion of the orthopedic insert. In other examples, a bone tunnel can be reamed to a greater or lesser degree or depth in order to configure the bone tunnel for insertion of an orthopedic insert therein to a desired depth.

After reaming of an exit portion of a bone tunnel, an inserter apparatus, such as, for example, the inserter apparatus 1400 shown in FIGS. 14-15D, can be utilized to insert or seat an orthopedic insert into the bone tunnel. As shown in the illustrated example, the inserter apparatus 1400 can include a nose portion 1402 (also referred to as a distal portion) configured to have an orthopedic insert mounted thereon and a stem portion 1404 (also referred to as a main body portion) extending from the nose portion 1402 along a longitudinal axis L2-L2 of the inserter apparatus 1400. In some examples, the stem portion 1404 can be a handle portion for operation of the inserter apparatus 1400.

In some examples, the stem portion 1404 includes a proximal end portion 1418. In some examples, the proximal end portion 1418 can be wider than a main body of the stem portion and include a surface 1422 on the back or proximal end. The surface 1422 (shown in FIG. 15D) may be configured to be tapped with a hammer to drive an orthopedic insert into a bone tunnel.

In some examples, the stem portion 1404 further includes an opposing distal end portion 1420 configured be coupled to, attached to, or to be integral with the nose portion 1402. As can be seen in FIG. 15C, the distal end portion 1420 can include a distal face 1424 disposed at or defining an angle aa relative to the longitudinal axis L2-L2 of the inserter apparatus 1400. In some examples, the angle aa is configured to be complementary or substantially complementary to an angle of a sloped rim of a head portion of an orthopedic insert (such as, for example, the angle c of the sloped rim 116 of the orthopedic insert 100). In certain examples, the angle aa can be equal or substantially equal to the angle of the sloped rim of the orthopedic insert measured relative to the longitudinal axis of the orthopedic insert. In some examples, the angle aa is in a range of 55° to 65° relative to the longitudinal axis L2-L2 of the inserter apparatus 1400. In another example, the distal face 1424 can be disposed at a 60° angle relative to the longitudinal axis L2-L2 of the inserter apparatus 1400. Thus, an orthopedic insert 100 can be positioned on the inserter 1400 with the rim 116 in contact with the angled distal face 1424.

As can be seen in FIG. 15C, the nose portion 1402 can include two members referred to hereinafter as spring prongs 1426 extending from the angled distal face 1424 and having a through slot or gap 1428 between them. In some examples, the spring prongs 1426 can be moveable between a compressed state and an expanded state, where the spring prongs 1426 are biased toward the expanded state. In the compressed state, the gap 1428 can be narrower and at least the distal ends of the spring prongs 1426 can be closer to each other, whereas, in the expanded state, the gap 1428 can be wider and the distal ends of the spring prongs 1426 can be farther apart from each other. In some examples, a distance bb between outer surfaces of the distal ends of the spring prongs 1426 in the expanded state is within a range of 1 mm to 8 mm, such as 1.5 mm to 7 mm, 2 mm to 6 mm, 2 mm to 5 mm, etc. In some examples, the distance bb between the distal ends of the spring prongs 1426 is decreased in the compressed state relative to the expanded state. For example, the distance bb in the compressed state can be in a range of in a range of 0.8 mm to 11 mm, such as 1.5 mm to 8 mm, 2 mm to 5 mm, etc. In some examples, an overall length cc (FIG. 15A) of the nose portion 1402 is within a range of 6 mm to 25 mm, such as 8 mm to 20 mm, 9 mm to 18 mm, 11 mm to 17 mm, etc.

As discussed above, the nose portion 1402 can be configured to have an orthopedic insert mounted thereon. In some examples, the nose portion 1402 is sized and shaped to be inserted through a head portion (for example, the head portion 104) and into or through a bore (for example, the bore 108) of an orthopedic insert (for example, the orthopedic insert 100). In some examples, the length cc of the nose portion can be greater than a length of an orthopedic insert (for example, greater than the length a in FIG. 4A). For example, the length cc can be in a range of 1 mm to 3 mm longer than a length of an orthopedic insert. In other examples, the length cc can be less than or equal to the length of an orthopedic insert.

Further, in some examples, the distance bb between the distal ends of the spring prongs 1426 in the expanded state is greater than a width or diameter of the bore of an orthopedic insert (for example, the diameter b shown in FIG. 4C). For example, the distance bb in the expanded state can be in a range of 0.01 mm to 0.1 mm, such as 0.02 mm to 0.8 mm, 0.03 mm to 0.06 mm, etc., greater than the diameter of the bore of an orthopedic insert. Additionally, the distance bb in the compressed state can be approximately equal to (or slightly less than) the diameter of the bore of an orthopedic insert. Stated differently, positioning an insert over the spring prongs 1426 can compress the spring prongs together, and the outward force of the spring prongs on the insert can help to retain the insert on the inserter. Accordingly, in some examples, the spring prongs 1426 are sized and shaped to be moved from the expanded state to the compressed state for insertion into a bore of an orthopedic insert, and biasing of the spring prongs 1426 toward the expanded state can enable the orthopedic insert to be releasably retained on the nose portion 1402 via the friction fit or receiving of the spring prongs 1426 within the bore.

In some examples, an operator can load an orthopedic insert onto the nose portion. In some examples, the inserter apparatus 1400 can be selected from an inserter apparatus set or kit or can be manufactured to a specified dimensions for use with a selected orthopedic insert. In other examples, the inserter apparatus 1400 can be provided with an orthopedic insert pre-loaded on the nose portion 1402 thereof.

Once an orthopedic insert is loaded onto the nose portion 1402, the inserter apparatus 1400 can be used to align and insert the orthopedic insert into the reamed opening of a bone tunnel. After alignment of the orthopedic insert with the bone tunnel, a force can be applied to the surface 1422 of the proximal end portion 1418 of the inserter apparatus 1400 (for example, using a hammer) to drive the orthopedic insert into the bone tunnel to the desired depth (for example, full insertion or partial insertion). During the insertion, the projections on the orthopedic insert can engage with the interior surface of the bone tunnel. In some examples, the engagement between projections on the shaft portion of the orthopedic insert and the interior surface of the bone tunnel can be sufficient to overcome frictional forces between the spring prongs 1426 and the insert, allowing the inserter to be withdrawn leaving the insert in place in the bone tunnel. For example, a position of the orthopedic insert within the bone tunnel can be maintained as the nose portion 1402 is withdrawn from the bore of the orthopedic insert. In other examples, the inserter apparatus can include a collapsing device or mechanism that can cause the spring prongs 1426 to move closer together for release and withdrawal of the inserter from the bore of the orthopedic insert.

### Exemplary Methods

Exemplary methods of implantation and use of the orthopedic inserts and systems disclosed herein are described throughout the application. One or more of these methods can be combined with the exemplary methods discussed below.

In some examples, a method of stabilizing a joint between a first bone and a second bone includes forming a tunnel through the first bone, the tunnel extending between a medial aspect of the first bone and a lateral aspect of the first bone, the tunnel comprising a first opening in the medial aspect of the first bone and a second opening in the lateral aspect of the first bone opposite the first opening. In some examples, the method includes inserting an orthopedic insert (such as, for example, one of the orthopedic inserts 100, 900, 1000, 1100) into one of the first opening or the second opening of the tunnel to a selected depth. In some examples, the method includes threading a suture through the tunnel and through the bore of the orthopedic insert, securing a first end of the suture to the first bone, and securing a second end of the suture to the second bone.

In some examples, the inserting of the orthopedic insert into the one of the first opening or the second opening of the tunnel to the selected depth comprises fully inserting the orthopedic insert such that an exterior face of the head is flush with an exterior surface of the first bone.

In other examples, the inserting of the orthopedic insert into the one of the first opening or the second opening of the tunnel to the selected depth comprises inserting a portion of the orthopedic insert into the tunnel such that an exterior face of the head is offset from an exterior surface of the first bone, and wherein a portion of the suture exiting the orthopedic insert is offset from the exterior surface of the first bone.

As discussed above, in some examples, the orthopedic inserts and associated systems and methods can be utilized in a lateral suture technique treatment of a knee joint, such as a human knee joint and/or a canine stifle joint. In some examples, the orthopedic inserts and associated systems and methods can be utilized in other joint reconstruction techniques and/or for treatment of other joints, such as those illustrated in FIGS. 2D-2G. In some examples, the orthopedic inserts and associated systems and methods can be utilized in treatment of humans or other animals, such as dogs, cats, or others.

In another example, an exemplary kit can include one or a plurality of orthopedic inserts such as any of the orthopedic inserts described herein. The kit can further comprise one or a plurality of reamers, such as any of the reamer examples described herein, having a size or sizes corresponding to the size(s) of the orthopedic insert(s). The kit can further comprise one or a plurality of inserters, such as any of the inserters described herein, having a size or sizes corresponding to the size(s) of the orthopedic insert(s). In some examples, an orthopedic insert can be pre-positioned on the inserter. The kit can also comprise any or all of suture, anchors, and/or screws for a selected joint reconstruction procedure such as a lateral suture repair. In some examples, the kit can be provided in a sterilized, prepackaged condition.

Any or all of the orthopedic inserts and associated surgical tools and methods described herein can provide a number of significant advantages over existing joint reconstruction hardware and techniques. For example, the inserter and associated surgical tools and methods described herein can protect one or more bones of a reconstructed joint from wear or abrasion caused a suture contacting the bone as the joint is flexed and extended. In another example, the orthopedic inserts and associated surgical tools and methods described herein can limit loss of tension on a suture in a reconstructed joint over time relative to existing joint reconstruction hardware and techniques. In another example, the orthopedic inserts and associated surgical tools and methods described herein can limit or prevent failure or rupture of a suture in a reconstructed joint. In another example, the orthopedic inserts and associated surgical tools and methods described herein can offset a suture from soft tissue in a reconstructed joint. In another example, the orthopedic inserts and associated surgical tools and methods described herein can prevent or limit or reduce pain to a patient caused by compression of soft tissue by a suture in a reconstructed joint over existing joint reconstruction hardware and techniques. In another example, the orthopedic inserts and associated surgical tools and methods described herein can provide stabilization of an orthopedic insert via barbs on an outer surface of a shaft of the insert. In another example, the orthopedic inserts and associated surgical tools and methods described herein can enable insertion of an orthopedic insert to a selected or desired depth within a bone tunnel.

In another example, the orthopedic inserts described herein can include an angled face that is contoured to match a curvature or a contour of a surface of a bone in a reconstructed joint. In another example, the orthopedic inserts described herein can limit or prevent protrusion of a head of the insert relative to the bone and/or tissue surrounding the bone. In another example, the orthopedic inserts described herein can provide a bearing surface to limit or prevent wear or abrasion on an orthopedic insert caused a suture contacting the insert as a reconstructed joint is flexed and extended. In another example, the orthopedic inserts described herein can include an at least partially spherical head that can provide improved stress distribution to a bone in a reconstructed joint during loading of the joint. In another example, the orthopedic inserts described herein can include barbs stabilize a position of the insert within a bone tunnel of a reconstructed joint.

The reamer and/or the inserter can allow the surgeon to control the depth of insertion of the orthopedic insert. For example, reaming the bone using only the distal portion of the cutter can facilitate seating the orthopedic insert with all or substantially all of the head above the bone surface. Advancing the larger diameter proximal portion of the cutter into the bone can create a counterbore around the bone tunnel, the depth of which can determine the proportion of the head that extends beyond the bone surface when the insert is seated. The surgeon can thus select an insertion depth of the head according to the particular anatomy of the patient.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims and equivalents of the recited features. We therefore claim as all that comes within the scope and spirit of these claims.

1. An orthopedic insert, comprising:
an insert body comprising a head, and a shaft extending from the head along a longitudinal axis of the insert body, wherein the insert body has a first end and a second opposing end, the head defining the first end of the insert body;
wherein the insert body comprises a bore extending through the head and the shaft along the longitudinal axis; and
wherein the shaft comprises an exterior surface and the insert body further comprises a plurality of projections extending from the exterior surface of the shaft, the projections being arranged on the shaft in a plurality of rows spaced apart along the longitudinal axis of the insert body, the projections having a height measured relative to the exterior surface of the shaft, wherein the height of the projections in a respective row increases in a direction along the longitudinal axis from the second end toward the first end of the insert body.

2. The orthopedic insert of claim 1, wherein each of the projections comprises a first surface perpendicular to the longitudinal axis of the insert body and a second surface angled relative to the longitudinal axis of the insert body, wherein the first surface is oriented toward the first end of the insert body and the second surface is oriented toward the second end of the insert body.

3. The orthopedic insert of claim 2, wherein an intersection between the first surface and the second surface of each of the projections forms an exterior edge of the projection, the exterior edge shaped and sized to engage with bone on an interior surface of a bone tunnel.

4. The orthopedic insert of claim 1, wherein:
the projections comprise outer edges; and
a length of the outer edges of the projections decreases in the direction along the longitudinal axis from the second end toward the first end of the insert body.

5. The orthopedic insert of claim 1, wherein the head comprises a rim surrounding an opening of the bore, wherein the rim is rounded from an interior surface of the bore to an exterior surface of the head.

6. The orthopedic insert of claim 1, wherein the head comprises a sloped rim that forms an angle with the longitudinal axis of the insert body such that the rim comprises an upper portion and a lower portion, the upper portion being farther from the second end of the insert body along the longitudinal axis than the lower portion.

7. The orthopedic insert of claim 6, wherein:
the sloped rim of the head comprises an apex;
the bore comprises an interior surface;
the head comprises an exterior surface;
the sloped rim at the apex comprises a curved surface extending from the exterior surface of the head to the interior surface of the bore; and
a first portion of the curved surface extending from the apex to the exterior surface of the head comprises a first radius, and a second portion of the curved surface extending from the apex to the interior surface of the bore comprises a second radius, wherein the second radius is greater than the first radius.

8. The orthopedic insert of claim 7, wherein the curved surface of the sloped rim at a point diametrically opposite the apex comprises a third radius, wherein the third radius is greater than the first radius and less than the second radius.

9. A method of stabilizing a joint between a first bone and a second bone utilizing the orthopedic insert of claim 1, comprising:
forming a tunnel through the first bone, the tunnel extending between a medial aspect of the first bone and a lateral aspect of the first bone, the tunnel comprising a first opening in the medial aspect of the first bone and a second opening in the lateral aspect of the first bone opposite the first opening;
inserting the orthopedic insert of claim 1 into one of the first opening or the second opening of the tunnel to a selected depth;
threading a suture through the tunnel and through the bore of the orthopedic insert;
securing a first end of the suture to the first bone; and
securing a second end of the suture to the second bone.

10. An orthopedic insert, comprising:
a head;
a shaft extending from the head along a longitudinal axis of the orthopedic insert; and
a bore extending through the head and the shaft along a longitudinal axis of the orthopedic insert and having a constant diameter from a first end to a second opposing end of the bore;
wherein the head comprises a rim surrounding an opening of the bore, and the rim is disposed at an angle in a range of 5° to 45° relative to the longitudinal axis of the orthopedic insert; and
wherein the rim comprises a curved surface extending from an interior surface at the first end of the bore to an exterior surface of the head.

11. The orthopedic insert of claim 10, wherein the rim is sloped and disposed at an angle relative to the longitudinal axis of the orthopedic insert such that the rim comprises an extended portion and a recessed portion, the extended portion being farther from the second end of the bore than the recessed portion.

12. The orthopedic insert of claim 11, wherein the sloped rim of the head comprises an apex; and
wherein a first portion of the curved surface extending from the apex to the exterior surface of the head comprises a first radius, and a second portion of the curved surface extending from the apex to the interior surface of the bore comprises a second radius that is greater than the first radius.

13. The orthopedic insert of claim 10, further comprising a plurality of annular ribs disposed on an exterior surface of the shaft.

14. The orthopedic insert of claim 13, wherein each of the plurality of annular ribs comprises a plurality projections circumferentially spaced apart from each other on a respective annular rib.

15. The orthopedic insert of claim 14, wherein the plurality of annular ribs are spaced apart from each other on the shaft along the longitudinal axis of the orthopedic insert, wherein each of the projections has a height measured relative to the exterior surface of the shaft, and wherein the height of the projections in a respective annular rib increases in a direction along the longitudinal axis from the second end toward the first end of the bore.

16. An orthopedic insert system, comprising:
an orthopedic insert comprising an insert body, the insert body comprising a head, a first shaft extending from the head, a bore extending through the head and the shaft along a longitudinal axis of the insert body, and a plurality of projections extending from an exterior surface of the first shaft, wherein the insert body has a proximal end and a distal end, the head defining the proximal end of the insert body, and wherein the projections are arranged on the first shaft in a plurality of rows spaced apart along the longitudinal axis of the insert body, the projections having a height measured relative to the exterior surface of the first shaft, wherein the height of the projections in a respective row decreases in a direction along the longitudinal axis from the proximal end toward the distal end; and
an inserter apparatus comprising a second shaft and a nose portion disposed at a distal end portion of the second shaft, wherein the nose portion comprises a pair of prongs that are movable between a compressed configuration and an expanded configuration, and wherein the pair of prongs are sized and shaped to be inserted through an opening of the bore at the head of the orthopedic insert, and wherein the pair of prongs, when in the expanded configuration, are configured to be friction fit within the bore.

17. The orthopedic insert system of claim 16, wherein the head of the orthopedic insert comprises an angled proximal face at the proximal end of the insert body, the angled proximal face disposed at a first angle relative to the longitudinal axis of the insert body, wherein the inserter apparatus further comprises an angled distal face of the second shaft disposed at a second angle relative to a longitudinal axis of the inserter apparatus, and wherein the angled distal face is proximal relative to the nose portion along the longitudinal axis of the inserter apparatus.

18. The orthopedic insert system of claim 17, wherein the first angle is complementary to the second angle such that the angled distal face of the inserter apparatus is configured to receive the angled proximal face of the insert body.

19. The orthopedic insert system of claim 17, wherein the first angle is 30° relative to the longitudinal axis of the insert body and the second angle is 60° relative to the longitudinal axis of the inserter apparatus.

20. The orthopedic insert system of claim 16, further comprising a reamer apparatus, the reamer apparatus comprising a third shaft, a reamer bit disposed at a distal end portion the third shaft, and a pilot guide distal of the reamer bit, wherein the pilot guide is configured to be seated within a bone tunnel, and wherein the reamer bit is configured to increase a diameter of a portion of the bone tunnel such that the orthopedic insert can be inserted into the bone tunnel.
